(19) United States
(12) Patent Application Publication
(10) Pub. No.: US 2006/0154331 A1
(43) Pub. Date: Jul. 13, 2006

(54) Erythrocyte differentiation factor, gene encoding same, and methods of use thereof

(76) Inventors: Nili Avidan, Rehovot (IL); Edna Ben-Asher, Tel Aviv (IL); Tavyia Olender, Rehovot (IL); Doron Lancet, Rehovot (IL); Jacques S Beckmann, Rehovot (IL); Hannah Tamary, Raanana (IL); Orly Dgany, Saba (IL)

Correspondence Address: Kathy Smith Dias;Heslin Rothenberg Farley & Mesiti, 5 Columbia Circle, Albany, NY, 12203, US

(21) Appl. No.: 10/530,117

(22) PCT Filed: Oct. 14, 2003
(86) PCT No.: PCT/IL03/00831

§ 371 Date: Nov. 2, 2005

(30) Foreign Application Priority Data
Oct. 15, 2002 (US) 60418165

(51) Int. Cl. A61K 38/18; C07K 14/475; C07H 21/04; C12P 21/06; C12N 5/08

(52) U.S. Cl. 435/69.1; 530/399; 536/23.5; 514/12; 435/372; 435/320.1

(57) ABSTRACT

The present invention relates to an erythrocyte differentiation factor designed Codanin-1, and to an isolated nucleic acid which encodes for the differentiation factor. Mutations in the differentiation factor are associated with Congenital Dyserythropoietic Anemias (CDA), a group of inherited red blood cell disorders associated with dysplastic changes in late erythroid precursors. The present invention further relates to uses of the differentiation factor to promote the differentiation of hematopoietic stem cells to erythrocytes, as well as for the treatment and diagnosis of anemia.

### FIELD OF THE INVENTION

The present invention relates to an erythrocyte differentiation factor, to a gene encoding the factor, and to uses thereof in promoting the differentiation of hematopoietic stem cells to erythrocytes. Mutations in the differentiation factor, designated Codanin-1, are associated with Congenital Dyserythropoietic Anemias (CDA), a group of inherited red blood cell disorders associated with dysplastic changes in late erythroid precursors.

### BACKGROUND OF THE INVENTION

Erythropoiesis, the production of red blood cells, occurs continuously to offset cell destruction. Erythropoiesis is a precisely controlled physiological mechanism enabling sufficient numbers of red blood cells to be available for proper tissue oxygenation, but not so many that the cells would impede circulation. The formation of red blood cells occurs in the bone marrow and is under the control of the hormone, erythropoietin. Erythropoietin is the primary humoral regulator of erythropoiesis and it supports the proliferation and terminal maturation of erythrocytes from hematopoietic stem cells.

Congenital dyserythropoietic anemias (CDA) constitute a rare group of inherited red blood cell disorders associated with morphological and functional abnormalities of erythropoiesis, namely with dysplastic changes in late erythroid precursors. CDAs have been classified into three types (I-III), with some patients still unassigned, whereas the most common form is the autosomal recessive CDA type II [MIM 224100], with more than 250 cases described to date (Iolascon et al. 2001). The disease gene maps to 20q11.2 in most studied families (Gasparini et al. 1997). The least common of the CDAs, the autosomal dominant CDAIII [MIM 105600], was localized upon linkage analysis of a large Swedish family to chromosome 15q22 (Lind et al. 1995).

CDA type I (CDAI) [MIM 224120, gene symbol CDAN1] is an autosomal recessive disease. CDAI patients present with moderate to severe macrocytic anemia. Bone marrow aspirates reveal binuclear intermediate and late erythroid precursors, as well as internuclear chromatin bridges. Ultrastructural erythroid features include spongy heterochromatin and invagination of the nuclear membrane, carrying cytoplasm and cytoplasmic organelles into the nucleus. Dysmorphic features, mainly syndactyly and the absence or hypoplasia of phalanges and nails, have also been observed in several patients. Arrest of DNA synthesis and apoptotic features in erythroid precursors were previously described (Tamary et al. 1996). Interferon α2 was incidentally found to ameliorate the anemia and to partially reverse the morphological abnormalities of the erythroid precursors, by an unknown mechanism.

Previously, a cluster of 45 Israeli Bedouin with CDAI enabled mapping of the CDAN1 disease gene to a 2 Mb interval between markers D15S779 and D15S778 on human chromosome 15q15 (Tamary et al. 1998).

U.S. Pat. No. 6,596,688 discloses use of peptides derived from human chorionic gonadotropin and derivatives and analogues thereof, for promoting hematopoiesis.

U.S. Pat. No. 5,786,323 discloses a combination of stem cell factor with gp130 signaling to support proliferation, differentiation and terminal maturation of erythroid cells from normal human hematopoietic stem cells.

U.S. Pat. No. 5,648,248 discloses methods for producing differentiated cells from immature hematopoietic cells comprising introducing a gene coding for a conditional v-rel estrogen receptor fusion protein, that causes estrogen-dependent but otherwise unaltered v-rel-specific transformation, into bone marrow cells.

U.S. Pat. No. 5,527,776 discloses a method of treating a subject with an anemia characterized by deficient total hemoglobin, said method comprising administering to the subject a complex comprising insulin like growth factor-1 (IGF-1) and insulin-like growth factor binding protein-3 (IGFBP-3) in an amount sufficient to improve the level of total hemoglobin.

Erythropoiesis plays an important role in hemostatic mechanism. Diseases involving abnormal differentiation/proliferation of red blood cells (such as CDA) are clinically dangerous. Therefore it is considered that isolation and identification of a factor, which modulates erythrocyte differentiation or proliferation, will be useful in the art. Furthermore, identification of the CDAN1 disease gene helps to clarify the pathogenesis of the CDA1 disease and may contribute to the development of novel strategies for its management, and may facilitate the cloning of the genes involved in other types of CDA.

### SUMMARY OF THE INVENTION

The present invention relates to an erythrocyte differentiation factor designated Codanin-1, having an amino acid sequence as set forth in at least one of SEQ ID NOS: 1-3 (FIG. 4), or a fragment, mutant or variant thereof. The present invention further relates to an isolated nucleic acid having a nucleotide sequence as set forth in SEQ ID NO: 4 (genomic clone AC090510), or a fragment, mutant or variant thereof, which encodes for the Codanin-1 differentiation factor. Mutations in the differentiation factor are associated with Congenital Dyserythropoietic Anemias (CDA), a group of inherited red blood cell disorders associated with dysplastic changes in late erythroid precursors. The present invention further relates to uses of the Codanin-1 differentiation factor to promote the differentiation of hematopoietic stem cells to erythrocytes. The present invention further relates to uses of the Codanin-1 differentiation factor for diagnosis or treatment of CDA and related diseases.

Thus, according to one embodiment, the present invention provides an erythrocyte differentiation factor having an amino acid sequence as set forth in at least one of SEQ ID NOS: 1-3 or a fragment, mutant or variant thereof.

According to another embodiment, the erythrocyte differentiation factor is designated Codanin-1, deposited with the Genebank Database under Accession No. AF525398. The present invention further provides a pharmaceutical composition comprising the erythrocyte differentiation factor, and a pharmaceutically acceptable carrier.

According to yet another embodiment, the erythrocyte differentiation factor is obtained from a non-mammalian cell. According to yet another embodiment, the erythrocyte differentiation factor is obtained from a mammalian cell. According to yet another embodiment, the erythrocyte differentiation factor is obtained from a human cell. According to yet another embodiment, the erythrocyte differentiation factor of the invention is glycosylated.

According to one embodiment, the erythrocyte differentiation factor promotes the differentiation of hematopoietic stem cells to erythrocytes. According to another embodiment, the differentiation factor promotes the proliferation of erythrocytes.

The present invention further provides an isolated nucleic acid encoding an erythrocyte differentiation factor, the isolated nucleic acid having a nucleotide sequence as set forth in SEQ ID NO: 4 (genomic clone AC090510), or a fragment, mutant or variant thereof.

According to one embodiment, the isolated nucleic acid is designated Codanin-1, deposited with the Genbank Database Accession No.: AF-525398. According to another embodiment, the present invention provides an expression vector comprising the isolated nucleic acid.

According to yet another embodiment, the isolated nucleic acid is obtained from a non-mammalian species. According to yet another embodiment, the isolated nucleic acid is obtained from a mammalian species. According to yet another embodiment, the isolated nucleic acid is obtained from a human source.

Furthermore, according to yet another embodiment, the present invention provides a method of promoting erythroid differentiation, comprising the step of culturing hematopoietic stem cells in a culture medium comprising an erythrocyte differentiation factor having an amino acid sequence as set forth in at least one of SEQ ID NOS: 1-3, or a fragment, mutant or variant thereof, in an amount effective to promote the differentiation of the hematopoietic stem cells to erythrocytes.

Furthermore, according to yet another embodiment, the present invention provides a method of producing erythrocytes in vitro, comprising the step of culturing hematopoietic stem cells in a culture medium comprising an erythrocyte differentiation factor having an amino acid sequence as set forth in at least one of SEQ ID NOS: 1-3, or a fragment, mutant or variant thereof, in an amount effective to induce differentiation of the hematopoietic cells, thereby producing erythrocytes.

Furthermore, according to yet another embodiment, the present invention provides a method of promoting erythroid differentiation in vitro, comprising the steps of: a) transfecting a culture of hematopoietic stem cells with a vector comprising an isolated nucleic acid encoding an erythrocyte differentiation factor, the isolated nucleic acid having a nucleotide sequence as set forth in SEQ ID NO: 4, or a fragment, mutant or variant thereof; and b) expressing the differentiation factor in the hematopoietic stem cells, wherein the differentiation factor is expressed in an amount effective to promote the differentiation of the hematopoietic stem cells to erythrocytes.

Furthermore, according to yet another embodiment, the present invention provides a method of producing erythrocytes in vitro, comprising the steps of: a) transfecting a culture of hematopoietic stem cells with a vector comprising an isolated nucleic acid encoding an erythrocyte differentiation factor, the isolated nucleic acid having a nucleotide sequence as set forth in SEQ ID NO: 4, or a fragment, mutant or variant thereof; and b) expressing the differentiation factor in the hematopoietic stem cells, wherein the differentiation factor is expressed in an amount effective to induce differentiation of the hematopoietic stem cells, thereby producing the erythrocytes.

The culture medium applied in the methods of the present invention for producing or promoting hematopoietic differentiation to erythrocytes, in vitro, may comprise other differentiation factors in addition to the erythrocyte differentiation factor of the invention. According to one embodiment, the culture medium further comprises an erythrocyte differentiation factor selected from the group consisting of: GMCSF, IL-3, and erythropoietin (EPO).

Furthermore, according to yet another embodiment, the present invention provides a method for producing erythrocytes in a subject, comprising the steps of: a) retrieving hematopoietic stem cells from the subject; b) contacting the hematopoietic cells of (a) with a culture medium comprising an erythrocyte differentiation factor derived from the same mammalian species as that of said subject, the differentiation factor having an amino acid sequence as set forth in at least one of SEQ ID NOS: 1-3 or a fragment, mutant or variant thereof; c) culturing the cells of (b) in vitro, to produce a cellular preparation, wherein culturing of said cells induces differentiation of the hematopoietic cells, thereby producing erythrocytes; and d) administering the cellular preparation of (c) to said subject.

Furthermore, according to yet another embodiment, the present invention provides a method for producing erythrocytes in a subject, comprising the steps of: a) removing hematopoietic stem cells from the subject; b) transfecting the hematopoietic stem cells with a vector comprising an isolated nucleic acid encoding an erythrocyte differentiation factor derived from the same mammalian species as that of said subject, the isolated nucleic acid having a nucleotide sequence as set forth in SEQ ID NO: 4, or a fragment, mutant or variant thereof; c) expressing the differentiation factor in the hematopoietic stem cells; d) culturing the cells in vitro, to produce a cellular preparation, wherein culturing induces differentiation of the hematopoietic stem cells, thereby producing erythrocytes; and e) administering the cellular preparation to said subject.

Furthermore, according to yet another embodiment the present invention provides a method for treating anemia in a subject comprising administering to the subject a pharmaceutical composition comprising a therapeutically effective amount of an erythrocyte differentiation factor derived from the same mammalian species as that of said subject, the erythrocyte differentiation factor having an amino acid sequence as set forth in at least one of SEQ ID NOS: 1-3, or a fragment, mutant or variant thereof.

Furthermore, according to yet another embodiment the present invention provides a method for treating anemia in a subject comprising administering a pharmaceutical composition comprising a construct encoding an erythrocyte differentiation factor derived from the same mammalian species as that of the subject, the erythrocyte differentiation factor having a polynucleotide sequence as set forth in SEQ ID NO: 4, or a fragment, mutant or variant thereof.

According to yet another embodiment, the present invention provides a vector comprising a polynucleotide sequence as set forth in SEQ ID NO: 4, or a fragment, mutant or variant thereof for purposes of cell therapy, in vivo. The vector may be stably integrated into host cell genomes. For these purposes, a preferred vector would be a viral vector, exemplified by but not limited to an adenovirus vector. The vector of the present invention may be also used for the purpose of gene therapy. Gene therapy may be accomplished by introducing a vector capable of expressing an erythrocyte differentiation factor having an amino acid sequence as set forth in at least one of SEQ ID NOS: 1-3 or a fragment, mutant or variant thereof, into a subject in need thereof.

According to yet another embodiment, the present invention provides a method of treating anemia by introducing cell transfected with the vectors of the present invention, to a subject in need thereof. The cells may be selected from a group of: somatic or hES cells. The cells of the invention may induce by cell therapy, methods as are known in the art, erythrocyte formation. Cell therapy methods as are known in the art comprise transplanting into an individual in need thereof cells that have been genetically engineered and/or selected to express the required function. The cells may be genetically modified or selected in vitro for cell lineages that express the required function, said cells being capable of expressing at least one of SEQ ID NOS: 1-3 or a fragment, mutant or variant thereof.

According to yet another embodiment, the present invention provides methods for increasing the expression of erythrocyte differentiation factor having an amino acid sequence as set forth in at least one of SEQ ID NOS: 1-3 or a fragment, mutant or variant thereof, in cells of an individual in need thereof by use of gene therapy techniques as known in the art.

Furthermore, according to another embodiment the present invention provides a method for diagnosing anemia in a subject comprising the step of analyzing in the subject a gene encoding an erythrocyte differentiation factor derived from the same mammalian species as that of said subject, the erythrocyte differentiation factor having a polynucleotide sequence as set forth in SEQ ID NO: 4, or a fragment, mutant or variant thereof.

According to one embodiment, the present invention provides an anti-codanin-1 antibody. According to another embodiment, the antibody of the invention is selected from the group consisting of: a full length antibody, an antibody having a human immunoglobulin constant region, a monoclonal IgG particularly of subclasses γ1 or γ4, a single chain antibody, an antibody fragment including, but not limited to, an F(ab′)₂ fragment or F(ab) or Fv, a labeled antibody, an immobilized antibody, an antibody conjugated with a heterologous compound. Preferably, the antibody of the invention is a polyclonal anti-codanin-1 antibody.

According to yet another embodiment, the present invention provides a method for diagnosing Congenital Dyserythropoietic Anemias in a subject, the method comprising: analyzing the level of codanin-1 in the subject by using an anti-codanin-1 antibody.

### BRIEF DESCRIPTION OF THE DRAWINGS

The present invention will be understood and appreciated more fully from the following detailed description taken in conjunction with the appended drawings which depict:

FIG. 1A is a physical map and genomic organization of the CDAN1 locus, wherein the relative positions of landmark microsatellite markers are indicated in the CDAI candidate interval defined by markers D15S779 and D15S778.

FIG. 1B is an expanded view of the 15 kb segment showing the genomic organization of CDAN1 and of two alternative transcripts isolated and sequenced from erythroid and fibroblast cells. Coding and non-coding exons are depicted as filled and open boxes, respectively.

FIG. 2A presents the pedigree of a Bedouin family with the segregation of the founder chromosome with the disease.

FIG. 2B is a sequence chromatogram from a wild type (WT) and diseased (M) genotype showing a C→T substitution in exon 25, converting arginine to tryptophan at codon 863.

FIG. 2C shows the segregation of the Bedouin mutation, as assayed by NcoI restriction wherein the mutation results in a gain of restriction site and, therefore, the restriction digest produced fragments of 175 and 237 bp, instead of the 412 bp band observed in healthy individuals.

FIG. 3 exhibits a Northern blot analysis for CDAN1 and β-Actin mRNAs exposed for 1 week and overnight respectively.

FIG. 4 presents multiple alignment of human codanin-1 (SEQ ID NO: 1) with its putative mouse (SEQ ID NO: 2) and fugu (SEQ ID NO: 3) orthologs wherein identical and similar residues in the multiple-alignment chart are represented by ‘*’, ‘:’ and ‘.’, standing for strong and weak conservation, respectively; Conserved BLOCKS are confined within boxes, shaded boxes are used for known block similarity; Arrows indicate positions of mutated residues, where circled and non-circled black arrows specify missense mutations within family I-VI and VII-IX respectively, white arrows specify null mutations; and a thin black line represents a split between exons.

### DETAILED DESCRIPTION OF THE PRESENT INVENTION

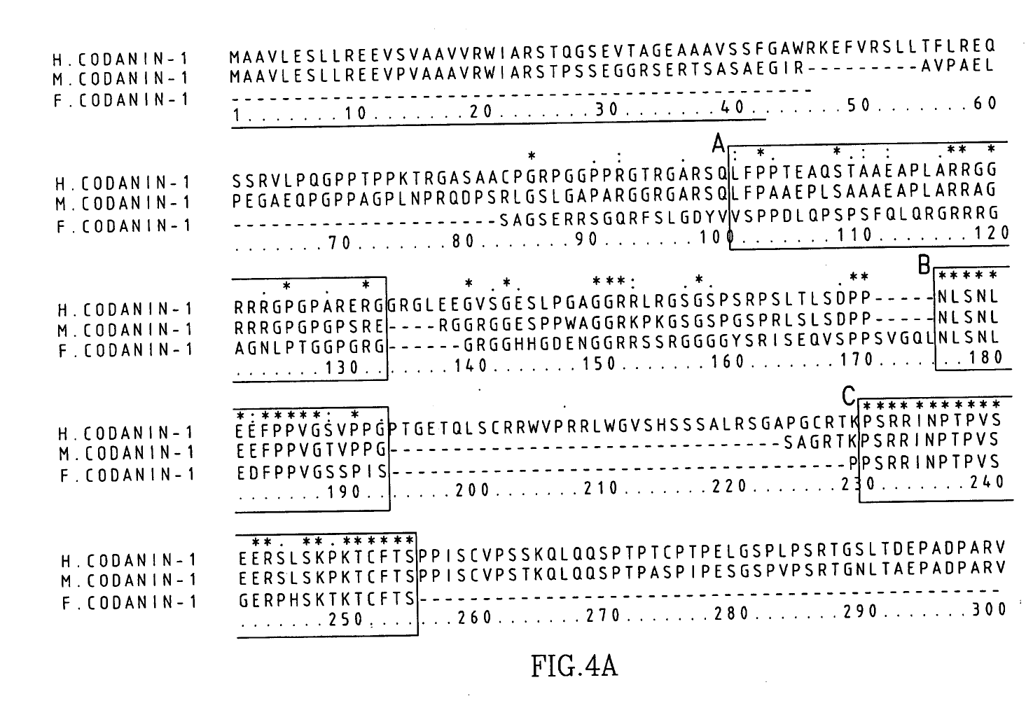

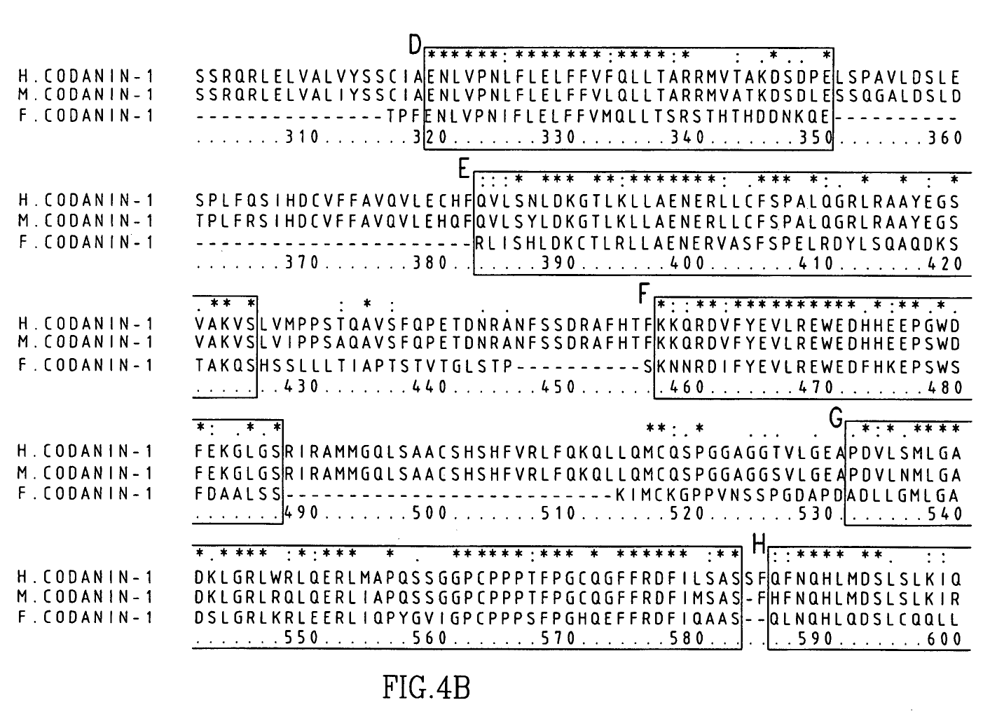

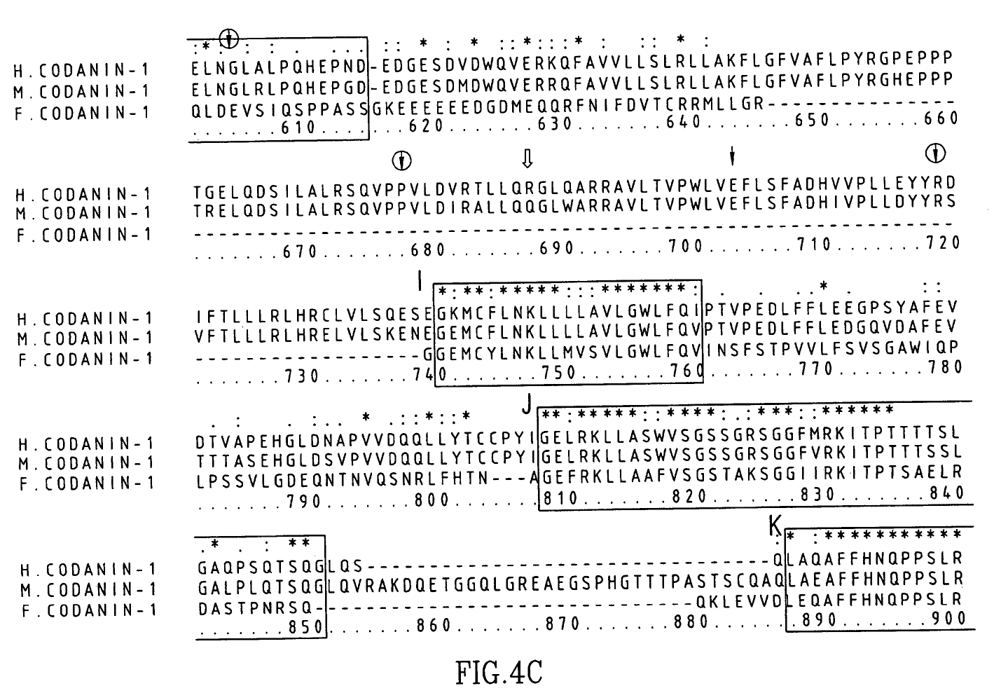

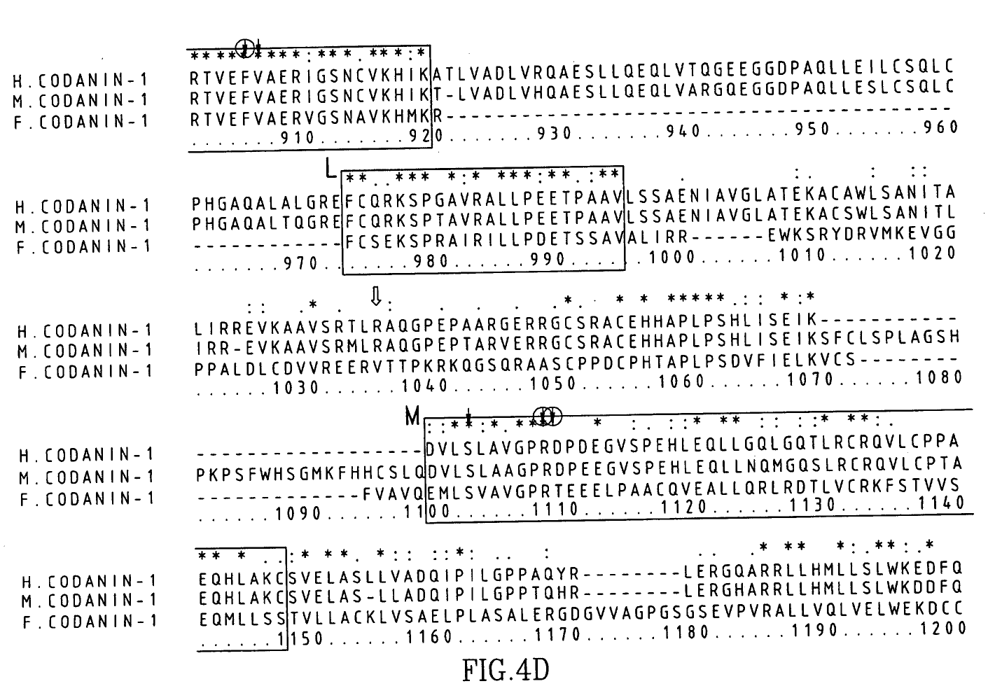

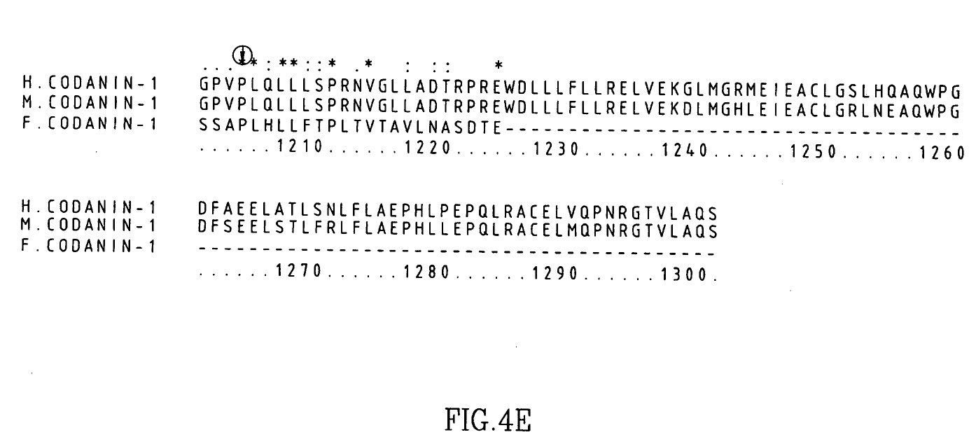

The present invention relates to an erythrocyte differentiation factor, designated Codanin-1, having an amino acid sequence as set forth in at least one of SEQ ID NOS: 1-3 (FIG. 4), or a fragment, mutant or variant thereof. The present invention further relates to an isolated nucleic acid having a nucleotide sequence as set forth in SEQ ID NO: 4 (genomic clone AC090510), or a fragment, mutant or variant thereof, which encodes for the differentiation factor. Mutations in the erythrocyte differentiation factor are associated with Congenital Dyserythropoietic Anemias (CDA), a group of inherited red blood cell disorders associated with dysplastic changes in late erythroid precursors. Thus, the present invention further relates to uses of the differentiation factor to promote erythrocyte differentiation and proliferation, and to produce erythrocytes.

As demonstrated herein, Applicants identified the CDAN1 gene, involved in Congenital Dyserythropoietic anemias type I (CDAI) through 12 different mutations in 9 CDAI families. This 28 exon gene, which encodes an ubiquitously expressed 4738 nucleotide mRNA was reconstructed based on gene prediction and homology searches. It encodes codanin-1, a putative o-glycosylated protein of 1226 amino acids, with no obvious transmembrane domains. Codanin-1 has a 150 residue amino-terminal domain with sequence similarity to collagens, and to two shorter segments that show weak similarities to the microtubule-associated proteins, MAP1B (neuraxin) and to synapsin. Without wishing to be bound by any particular concept or mechanism of action, these findings, and the cellular phenotype of the CDAI disease, suggest that codanin-1 may be involved in nuclear envelope integrity, conceivably related to microtubule attachments.

Thus, according to one embodiment, the present invention provides an erythrocyte differentiation factor having an amino acid sequence as set forth in at least one of SEQ ID NOS: 1-3, or a fragment, mutant or variant thereof. According to another embodiment, the erythrocyte differentiation factor is designated Codanin-1, deposited with the Genebank Database under Accession No. AF525398.

The present invention further provides a pharmaceutical composition comprising the erythrocyte differentiation factor, and a pharmaceutically acceptable carrier. The pharmaceutical composition is preferably used as a medicament for diseases involving abnormal erythrocyte differentiation, for example and without being limited to—Congenital Dyserythropoietic Anemias (CDA), and specifically for CDA type 1.

The present invention further provides a gene coding for the Codanin-1 erythrocyte differentiation factor of the present invention. Thus, according to one embodiment, the present invention provides an isolated nucleic acid encoding the erythrocyte differentiation factor of the present invention, the isolated nucleic acid having a nucleotide sequence as set forth in SEQ ID NO: 4, or a fragment, mutant or variant thereof. According to another embodiment, the isolated nucleic acid is designated Codanin-1, deposited with the Genebank Database under the Accession No. AF525398.

The present invention further provides a pharmaceutical composition comprising the isolated nucleic acid encoding the erythrocyte differentiation factor of the present invention, and a pharmaceutically acceptable carrier. The pharmaceutical composition is preferably used as a gene therapy for diseases involving abnormal erythrocyte differentiation, for example and without being limited to—in gene therapy of Congenital Dyserythropoietic Anemias (CDA), and specifically for CDA type 1.

According to yet another embodiment, the present invention provides an expression vector comprising the isolated nucleic acid encoding Codanin-1. An addition or improvement of a signal sequence, choice of host-vector system, and improvement of expression regulatory region may provide efficient expression. In addition, a host may be chosen to provide a glycosylated product.

An “expression vector” refers to a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. The term “operably linked” indicates that the segments are arranged so that they function in concert for their intended purposes, e.g. transcription initiates in the promoter and proceeds through the coding segment to the terminator. The term “expression vector”, refers to viral expression systems, autonomous self-replicating circular DNA, plasmid, and includes both expression and nonexpression plasmids. Where a recombinant microorganism or cell culture is described as hosting an “expression vector,” this includes both extrachromosomal circular DNA and DNA that has been incorporated into the host chromosome(s). Where a vector is being maintained by a host cell, the vector may either be stably replicated by the cells during mitosis as an autonomous structure, or is incorporated within the host's genome.

A “gene” is a segment of chromosomal DNA that encodes a polypeptide chain. A gene includes one or more regions encoding amino acids, which in some cases are interspersed with non-coding “intervening sequences” (“introns”), together with flanking, non-coding regions which provide for transcription of the coding sequence.

A “nucleic acid” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5N to 3N direction along the nontranscribed strand of DNA (i.e., the strand having a sequence homologous to the mRNA).

As defined herein an “isolated” or “substantially pure” nucleic acid (e.g., an RNA, DNA or a mixed polymer) is one which is substantially separated from other cellular components which naturally accompany a native human sequence or protein, e.g., ribosomes, polymerases, many other human genome sequences and proteins. The term embraces a nucleic acid sequence or protein, which has been removed from its naturally occurring environment, and includes recombinant or cloned DNA isolates and chemically synthesized analogs or analogs biologically synthesized by heterologous systems.

The term “plasmid” refers to an autonomous circular DNA molecule capable of replication in a cell, and includes both the expression and nonexpression types. Where a recombinant microorganism or cell culture is described as hosting an “expression plasmid”, this includes latent viral DNA integrated into the host chromosome(s). Where a plasmid is being maintained by a host cell, the plasmid is either being stably replicated by the cells during mitosis as an autonomous structure or is incorporated within the host's genome.

As hosts, both the prokaryote and eukaryote can be used. As prokaryote, bacteria such as Escherichia coli the genus Bacillus, for example, B. subtilis and the like can be used. As eukaryote, yeast such as the genus Saccharomyces, for example, S. serevisiae, insect cells such as Spodoptera frugiperda cells, Cabbage looper cells, Bombyx mori cells, animal cells such as human cells, monkey cells, mouse cells and the like can be used. Moreover, insects per se, such as Bombyx mori may be used.

As expression vectors, plasmid, phage, phagemid, virus such as baculo virus, vaccinia virus or the like can be used. A promoter in an expression vector is selected depending on host used. For example, lac promoter, trp promoter and the like can be used as bacterial promoters, and adhl promoter, pqk promoter and the like can be used as yeast promoters. On the other hand, baculo virus polyhedrin promoter can be used as insect promoter, and Simian virus 40 early or late promoter can be used for animal cells.

Transformation of a host with an expression vector can be carried out according to conventional procedures well known in the art, and these procedures are described in, for example, Current Protocols in Molecular Biology, John Wiley & Sons. Culturing of a transformant also can be carried out according to a conventional procedure.

Purification of the Codanin-1 differentiation factor from a culture or an organism can be carried out according to conventional procedures used for isolation and purification of a protein, for example, ultrafiltration, various types of column chromatography such as Q-Sepharose column chromatography and the like.

As contemplated herein analogs, fragments, mutants, substitutions, synthetics, variants of the isolated nucleic acid encoding Codanin-1, and of the polypeptide product of the isolated nucleic acid, are also included within the scope of the present invention.

Mutations can be made in a nucleic acid encoding the Codanin-1 differentiation factor such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. In one embodiment, the present invention should be considered to include sequences containing conservative changes which do not significantly alter the activity or binding characteristics of the resulting protein. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point. Further, other positions may be varied by themselves as long as the antigenic binding ability of the polypeptide is not destroyed.

The source of, the isolated nucleic acid encoding the Codanin-1 differentiation factor of the present invention, and of the polypeptide product of the nucleic acid, i.e. the Codanin-1 differentiation factor varies. In one embodiment, the nucleic acid encoding Codanin-1 may be obtained from a mammalian cell. In another embodiment, the nucleic acid may obtained from a human cell. Likewise, the Codanin-1 differentiation factor may be obtained from a mammalian cell. In another embodiment, the Codanin-1 differentiation factor may be obtained from a human cell.

As contemplated herein, the Codanin-1 erythrocyte differentiation factor is involved in the proliferation/differentiation of red blood cells (erythrocytes). In one embodiment, Codanin-1 promotes the differentiation of hematopoietic stem cells to erythrocytes. Thus, in one embodiment, the present invention provides a method of promoting differentiation of hematopoietic cell to erythrocytes, comprising the step of culturing hematopoietic stem cells in a culture medium comprising the Codanin-1 differentiation factor of the present invention, in an amount effective to promote the differentiation of the hematopoietic stem cells to erythrocytes.

Furthermore, in another embodiment, the present invention provides a method of producing erythrocytes in vitro, comprising the step of culturing hematopoietic stem cells in a culture medium comprising the Codanin-1 differentiation factor of the present invention, in an amount effective to induce differentiation of the hematopoietic cells, thereby producing erythrocytes.

Furthermore, in another embodiment, the present invention provides a method for producing erythrocytes in a subject, comprising the steps of: a) removing hematopoietic stem cells from the subject; b) contacting the hematopoietic cells with a culture medium comprising the Codanin-1 differentiation factor of the present invention; c) culturing the cells in vitro, to produce a cellular preparation; wherein culturing of the cells induces differentiation of the hematopoietic cells, thereby producing erythrocytes; and d) administering the cellular preparation to the subject.

Furthermore, in another embodiment, the present invention provides a method of promoting differentiation of hematopoietic cell to erythrocytes in vitro, comprising the steps of: a) transfecting a culture of hematopoietic stem cells with a vector comprising an isolated nucleic acid encoding an erythrocyte differentiation factor, the isolated nucleic acid having a nucleotide sequence as set forth in SEQ ID NO: 4, or a fragment, mutant or variant thereof; and b) expressing the differentiation factor in the hematopoietic stem cells; wherein the differentiation factor is expressed in an amount effective to promote the differentiation of the hematopoietic stem cells to erythrocytes.

Furthermore, in another embodiment, the present invention provides a method of producing erythrocytes in vitro, comprising the steps of: a) transfecting a culture of hematopoietic stem cells with a vector comprising an isolated nucleic acid encoding an erythrocyte differentiation factor, the isolated nucleic acid having a nucleotide sequence as set forth in SEQ ID NO: 4, or a fragment, mutant or variant thereof; and b) expressing the differentiation factor in the hematopoietic stem cells; wherein the differentiation factor is expressed in an amount effective to induce differentiation of the hematopoietic stem cells, thereby producing the erythrocytes.

Furthermore, in another embodiment, the present invention provides a method for producing erythrocytes in a subject, comprising the steps of: a) removing hematopoietic stem cells from the subject; b) transfecting the hematopoietic stem cells with a vector comprising an isolated nucleic acid encoding an erythrocyte differentiation factor derived from the same mammalian species as that of the subject, the isolated nucleic acid having a nucleotide sequence as set forth in SEQ ID NO: 4, or a fragment, mutant or variant thereof; c) expressing the differentiation factor in the hematopoietic stem cells; d) culturing the cells in vitro, to produce a cellular preparation; wherein culturing induces differentiation of the hematopoietic stem cells, thereby producing erythrocytes; and e) administering the cellular preparation to the subject.

According to yet another embodiment, the present invention provides a vector comprising a polynucleotide sequence as set forth in SEQ ID NO: 4, or a fragment, mutant or variant thereof for purposes of cell therapy, in vivo. The vector may be stably integrated into host cell genomes. For these purposes, a preferred vector would be a viral vector, exemplified by but not limited to an adenovirus vector. A preferred embodiment of the expression vectors is viral vectors, for example: adenoviruses, retroviruses or lentiviruses. The use of adenovirus vectors is known in the art. The use of SV-40 derived viral vectors and SV40 based packaging systems has been described by Fang et al. (Anal Biochem. 1997, 254:139-43)

When using viruses as vectors, the viral surface proteins are generally used to target the virus. As many viruses, such as adenoviruses, are somewhat unspecific in their cellular tropism, it may be desirable to impart further specificity by using a cell-type or tissue-specific promoter. For the purpose of the present invention the promoter activity may be controlled by factors specifically abundant in the hemapoietic system.

Alternatively, the viral vector may be engineered to express an additional protein on its surface, or the surface protein of the viral vector may be changed to incorporate a desired sequence. The viral vector may thus be engineered to express one or more additional epitopes which may be used to target said viral vector. For instance, cytokine epitopes, MHC class II binding peptides, or epitopes derived from homing molecules may be used to target the viral vector in accordance with the teaching of the invention.

The vector of the present invention may be used for the purpose of gene therapy by introducing a vector capable of expressing an erythrocyte differentiation factor having an amino acid sequence as set forth in at least one of SEQ ID NOS: 1-3 or a fragment, mutant or variant thereof, into a subject in need thereof. Methods for gene delivery to cells are known in the art, for example, U.S. Pat. No. 6,448,083.

According to yet another embodiment, the present invention provides a method of treating anemia by introducing cell transfected with the vectors of the present invention, to a subject in need thereof. The cells may be selected from a group of: somatic cells and hES cells. The cells are obtained from cord blood or from a suitable donor. The cells of the invention may induce by cell therapy, methods as are known in the art, erythrocyte formation. Cell therapy methods as are known in the art comprise transplanting into an individual in need thereof cells that have been genetically engineered and/or selected to express the required function. The cells may be genetically modified or selected in vitro for cell lineages that express the required function, said cells being capable of expressing at least one of SEQ ID NOS: 1-3 or a fragment, mutant or variant thereof.

According to yet another embodiment, the present invention provides methods for increasing the expression of erythrocyte differentiation factor having an amino acid sequence as set forth in at least one of SEQ ID NOS: 1-3 or a fragment, mutant or variant thereof, in cells of an individual in need thereof by use of gene therapy techniques as known in the art.

The methods of the present invention may be applied in combination with other methods known in the art for stimulating erythrocyte differentiation. Thus, the culture medium for culturing hematopoietic stem cells with the Codanin-1 differentiation factor of the present invention, may further include any factor known in the art for stimulating erythrocyte differentiation such as, GMCSF, IL-3, and erythropoietin. The amount of each factor in the culture medium is an amount effective to promote or induce the differentiation of the hematopoietic stem cells to erythrocytes. Factor known in the art for inducing erythrocyte differentiation may be also added to the culture medium of hematopoietic stem cells transfected with a vector comprising an isolated nucleic acid encoding an erythrocyte differentiation factor derived from the same mammalian species as that of the subject, the isolated nucleic acid having a nucleotide sequence as set forth in SEQ ID NO: 4 (genomic clone AC090510).

For use in promoting differentiation of hematopoietic cell to erythrocytes in vivo, the differentiation factor may be administered in a pharmaceutical composition by any suitable route, including orally, parentally, by inhalation spray, rectally, transdermally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally.

The terms “pharmaceutical preparation” or “pharmaceutical composition” are used herein interchangeably to describe an amount of the differentiation factor of the present invention, and a pharmaceutically acceptable carrier. Such preparations may routinely contain salt, buffering agents, preservatives, compatible carriers, and optionally other therapeutic agents. The salts should be pharmaceutically acceptable or may conveniently be used to prepare pharmaceutically acceptable salts thereof and are not excluded from the scope of the invention. Acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts.

The differentiation factor of the invention or compositions thereof can be administered alone or in combination with other therapeutic agents. For example, treatment with differentiation factor of the invention can be combined with traditional therapies for inducing erythrocyte differentiation.

According to one embodiment, the present invention provides an anti-codanin-1 antibody. According to another embodiment, the antibody of the invention is selected from the group consisting of: a full length antibody, an antibody having a human immunoglobulin constant region, a monoclonal IgG particularly of subclasses γ1 or γ4, a single chain antibody, an antibody fragment including, but not limited to, an F(ab′)₂ fragment or F(ab) or Fv, a labeled antibody, an immobilized antibody, an antibody conjugated with a heterologous compound. Preferably, the antibody of the invention is a polyclonal anti-codanin-1 antibody.

According to yet another embodiment, the present invention provides a method for diagnosing Congenital Dyserythropoietic Anemias in a subject, the method comprising: analyzing the level of codanin-1 in the subject by using an anti-codanin-1 antibody.

According to one embodiment, pharmaceutical preparation is orally administered in solid or liquid dosage form. In another embodiment, the pharmaceutical preparation is intravenously, intraarterially, subcutaneously, intradermally, intraperitoneally, intramuscularly or intralesionally injected in liquid form. In another embodiment, the pharmaceutical preparation is a pellet, a tablet, a capsule, a solution, a suspension, an emulsion, a gel, a cream, a suppository, or a parenteral formulation.

As used herein a “pharmaceutically acceptable carrier” may be a solid carrier for solid formulations, a liquid carrier for liquid formulations, or mixtures thereof. Solid carriers include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof. For liquid formulations, pharmaceutically acceptable carriers may be aqueous or non-aqueous solutions, suspensions, or emulsions. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Carrier formulation suitable for oral, subcutaneous, intravenous, intramuscular and other administrations can be found in Remington's Pharmaceutical Sciences, Mack Publishing Co., Easton, Pa.

Parenteral vehicles (for subcutaneous, intravenous, intraarterial, intraperitoneal or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions.

In addition, the compositions may further comprise binders (e.g. cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCl, acetate, phosphate) of various pH and ionic strength, additives such as gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, chlorobutanol, parabens and thimerosal), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

In one embodiment, the pharmaceutical compositions provided herein are controlled release compositions, i.e. compositions in which the differentiation agent is released over a period of time after administration. In another embodiment, the composition is an immediate release composition, i.e. a composition in which all of the differentiation agents are released immediately after administration.

The preparation of pharmaceutical compositions which contain an active component is well understood in the art, for example by mixing, granulating, or tablet-forming processes. The active therapeutic ingredient is often mixed with excipients which are pharmaceutically acceptable and compatible with the active ingredient. For oral administration, the differentiation factor is mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. For parenteral administration, the differentiation factor is converted into a solution, suspension, or emulsion, if desired with the substances customary and suitable for this purpose, for example, solubilizers or other.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXPERIMENTAL DETAILS SECTION

### Example 1

### Identification and Mapping of the CDAN1 Gene

Previously a cluster of 45 highly inbred Israeli Bedouin with CDAI enabled the mapping of the CDAN1 disease gene to a 2 Mb interval on human chromosome 15q15 (Tamary et al.-1998). Applicants have presently refined this interval to a 1.2 Mb interval, containing 15 candidate genes as detailed herein.

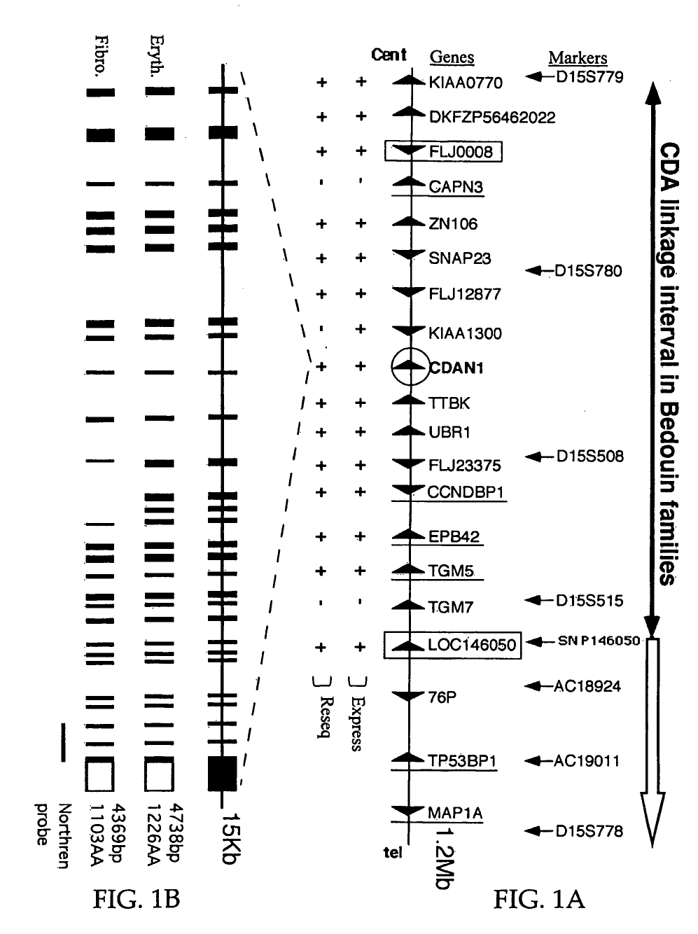

All patients showed a similar clinical picture and in all the subjects the diagnosis was confirmed by bone marrow electron microscopy. DNA was extracted from whole blood and RNA was extracted from the diagnostic bone marrow aspiration. All studies were approved by the institutional review board of Rabin Medical Center. Using new polymorphic markers from genomic clones (AC019011, AC018924), and an informative SNP within the defined transcript for the putative transcription factor LOC146050, Applicants refined the critical CDAI interval to 1.2 Mb (FIG. 1A).

In an attempt to identify the underlying mutations, a systematic in silico analyses through gene prediction and homology searches of the critical CDAI interval was conducted, and 17 transcripts or putative genes were found. The physical map and genomic organization of the CDAN1 locus, wherein the relative positions of landmark microsatellite markers are indicated in the CDAI candidate interval defined by markers D15S779 and D15S778 is shown in FIG. 1A. The CDA1 interval is indicated by black and white arrows. An informative SNP within the newly defined transcript LOC146050 (under the white arrow) enabled to restrict the linkage interval. The genes and transcripts identified are boxed: known genes are underlined, unknown transcripts are unboxed, inferred genes in within square frames and CDAN1 is circled. CDAN1 exons are based on two partial transcripts, DKFZp434G2127 and B185513 or gene prediction and RT-PCR (FIG. 1B). The lengths of the corresponding transcripts and inferred proteins are indicated to the right.

Fifteen genes prevailed as potential candidates, based on their expression as determined by RT-PCR in erythrocytes grown in liquid culture (Pope et al. 2000), including two redefined and two newly characterized transcripts, namely UBR1 (AF525401), TTBK (AF525400), FLJ008 (AF525397) and LOC146050 (AF525399). The remaining two genes, Calpain3 (CAPN3) and TGM7 were not considered, as they are not expressed in erythrocytes. The predicted exons were amplified from genomic DNA or from mRNA of erythroid precursors of one Bedouin CDAI patient and of his healthy brother. These were subjected to sequence verification and mutation detection. Such systematic and comprehensive PCR and sequencing analyses was carried out for all coding regions of 14 out of the 15 candidate genes (excluding KIAA1300; FIG. 1A). The expression patterns of these genes were tested by RT-PCR on RNA from erythroid precursor cells (Pope et al. ibid) and are marked by a (+) or a (−) sign in the Express lane of FIG. 1A. Resequenced genes are marked by a + or − sign in the Reseq lane of that figure. All PCR reactions were performed according to standard procedures, and the products were subsequently sequenced using dye terminators of Big-Dyes kits on an ABI 3100 sequencer (Perkin-Elmer/Applied biosystems). Sequence comparisons were performed using the STADEN package and Sequencher™ software (Gene Codes cooperation, Ann Arbor, Mich., USA).

While in the first 13 genes tested no segregating mutation was identified, a homozygous mutation was identified in the Bedouin patient in the 14^{th} gene scrutinized (Table 1). This gene was reconstructed based on prediction programs and homology searches using two partial transcripts and two EST sequences (FIG. 1B). The gene, thus identified as CDAN1, has 28 exons spanning 15 kb of genomic DNA and it encompasses a putative 4738 nucleotide-long mRNA encoding a protein of 1226 amino acids, named herein by the Applicants Codanin-1, for congenital dyserythropoietic anemias type I.

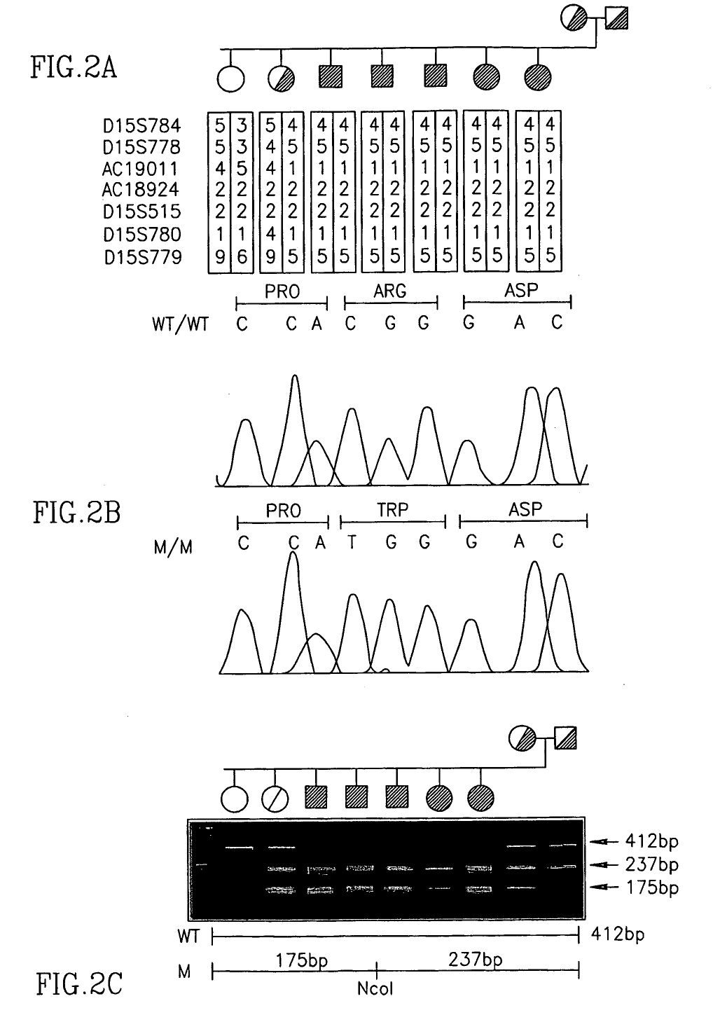

Subsequently 11 additional mutations in 8 other CDA I patient groups were identified in this gene (Table 1). A single homozygous C to T substitution in exon 24, converting arginine to tryptophan at codon 1040 and creating an NcoI restriction site, was found in all 45 CDAI Bedouin patients analyzed (FIG. 2C). A 412 bp PCR product was generated between the F and R primers wherein the mutation results in a gain of restriction site and, therefore, the restriction digest produced fragments of 175 and 237 bp, instead of the 412 bp band observed in healthy individuals.

A total of 184 chromosomes from healthy non-related Bedouin individuals were analyzed as controls for the Bedouin mutation (Table 1). For each of the additional mutations, close to 200 chromosomes from unrelated control individuals were analyzed. Three carriers, each for one mutation, were found. This is in accordance with the estimated frequency of CDAI heterozygotes in the European population. One of 184 Israeli Arab and two of 192 independent Bedouin control chromosomes were found to be carriers of this mutation (Table 1). Subsequently, two other CDAI patients from two unrelated families were found to be homozygous for two additional CDAN1 missense mutations: A proline to leucine substitution at codon 1129, found in a French Polynesian patient, and an asparagine to serine substitution at codon 598, present in three affected brothers from a family of French origin (Table 1). It is noteworthy that the three sibs also suffer from sensorineural deafness and lack of motile sperm cells. These conditions can be accounted for by a large 70 kb deletion 1 Mb distal to the CDAN1. Hence discovery of the A to G substitution in exon 12 indicates that these brothers carry, in addition to the 70 kb deletion, a missense mutation in CDAN1 responsible for the CDAI phenotype. It is surprising to find tightly linked independent mutations on the same haplotype. However, cases of contiguous gene syndromes have been reported (see, for example, Shanske et al. 2001), and given the growing number of mapped genetic disorders, such cases may be frequent enough to be encountered.

Three sporadic European CDAI patients were found to be compound heterozygotes for CDAN1 mutations. One patient has two distinct missense mutations, while the other two have one null and one missense mutation each (Table 1). Segregation analysis of each mutation in their respective families and genotyping control chromosomes was done by restriction fragment analysis (NEBiolabs) or by mass spectrometry SNP scoring (Sauer et al. 2002). Segregation analysis demonstrated that all patients inherited one mutation from each parent, thus confirming that the patients are true compound heterozygotes. In addition two patients of European descent and one of Arab descent were shown to carry one distinct missense mutation each (Table 1). The unidentified mutations may be located in the promoter or introns of CDAN1, or in some as yet unidentified exons. It is of interest to note that in one additional patient (diagnosed by AI), who presented the clinical characteristics of CDAI, was merely found to be a compound heterozygote for two synonymous nucleotide changes (data not shown). For this CDAI patient, pathogenic mutations remain to be identified in CDAN1, or else in a second locus involved in the etiology of this disease. In any event, the data presented herein imply a considerable molecular homogeneity for CDAI.

### Example 2

### Northern Blot Analysis

Northern blot analysis was performed with a cDNA probe for exons 26-28 on RNA from 8 different human tissues. The membrane was purchased from Clontech (BD Biosciences-Clontech) and was hybridized according to the manufacturer's instructions. The membrane was first probed with codanin-1 (Exons 26-28, FIG. 1B) and then subsequently reprobed with β-Actin cDNA as an internal control.

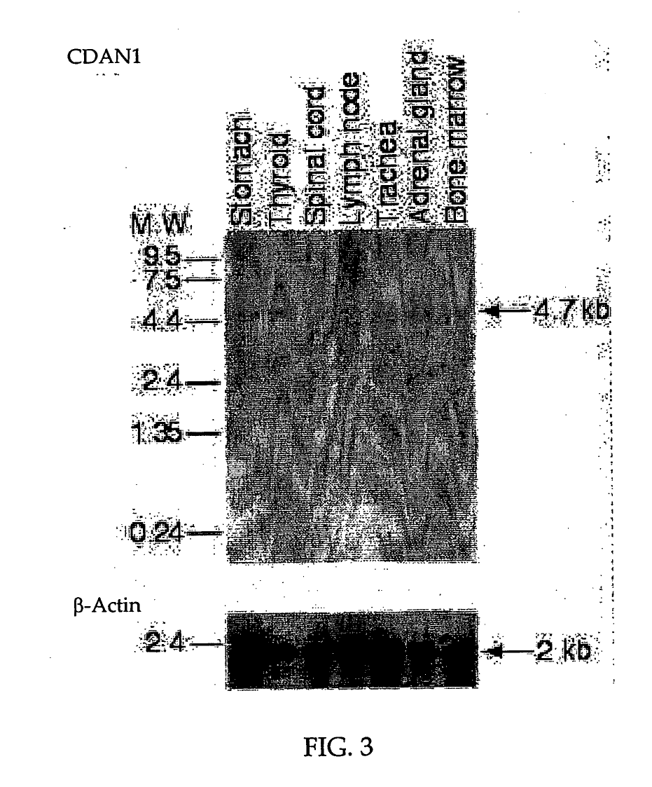

All tissues expressed the same 4.7 Kb band (FIG. 3A), suggesting that the gene is ubiquitously expressed and that the inferred mRNA is close to full length. A 369 bp shorter, alternatively spliced variant was found upon RT-PCR (data not shown) to be co-expressed in fibroblast cell line cultures from both CDAI patients and healthy controls, but not in erythrocytes. It is generated by in-exon splicing from exons 11 to exon 14 (FIG. 1B), that preserves the original reading frame. This mRNA isoform encodes a putative protein of 1103 amino acids. This major CDAN1 mRNA appears to be expressed at a level at least tenfold lower than β-Actin control as evidenced by their relative exposure times (FIG. 3B). This observation is in agreement with the results of expression array experiments (data not shown) and the low number of CDAN1 ESTs present in the databases.

### Example 3

### Multiple Alignment of Human Codanin-1

BLAST homology searches against several genomic sequence databases showed no obvious human codanin-1 paralog (SEQ ID NO: 1), but revealed two putative orthologs: one in the mouse syntenic region (84% identity) (SEQ ID NO: 2), and one in Fugu rubripes (44% identity) (FIG. 4) (SEQ ID NO: 3). Applicants also identified a putative ortholog in Drosophila melanogaster, (AF487678S2), which shares 23% identity with codanin-1. This protein, vanaso, has been proposed to be is involved in the fly's olfactory behavior, and is unlikely to be a functional CDAN1 ortholog. Codanin-1 has no clear intracellular location-specific domains, such as a trans-membrane or a signal peptide segments, it contains, however, numerous o-glycosylation consensus sites. In addition, the amino-terminal domain shares a significant homology with fibrillar collagens as revealed by FASTA (Pearson and Lipman, 1988) (FIG. 4). A multiple alignment was performed using the CLUSTALX program with the default parameters (Higgins et al. 1996). Further domain analysis was performed by

TABLE 1
CODANIN-I mutations in CDAI patients

| Family No. | Family | | | Nucleotide | Amino acid | | Detection |
|---|---|---|---|---|---|---|---|
| and ID | origin | Genotype | Exon | position^{a} | change | Frequency | method^{b} |
| I (5) | Israeli | homozygote | 24 | 3238C→T | Arg→Trp | 3/376 | +NcoI |
| | Bedouin | | | | (R1040W) | | |
| II (1) | French | homozygote | 26 | 3503C→T | Pro→Leu | 0/142 | −AciI |
| | Polynesia | | | | (P1129L) | | |
| III (1) | European | homozygote | 12 | 1910A→G | Asn→Ser | 0/196 | +DdeI |
| | | | | | (N598S) | | |
| IV (1) | European | Compound | 14 | 2129C→T | Pro→Leu | 1/196 | −Msp I |
| | | heterozygote | | | (P671L) | | |
| | | | 19 | 2716T→A | Phe→Ile | 0/170 | MALDI- |
| | | | | | (F866I) | | TOF |
| V (1) | European | Compound | 14 | 2158C→T | Arg→stop | 0/200 | +BtsI |
| | | heterozygote | | | (R680X) | | |
| | | | 14 | 2254C→T | Arg→Trp | 1/164 | +BsrI |
| | | | | | (R712W) | | |
| VI (1) | European | Compound | 23 | 3106C→T | Arg→stop | 0/196 | −TaqI |
| | | heterozygote | | | (R996X) | | |
| | | | 24 | 3242A→T | Asp→Val | 0/180 | MALDI- |
| | | | | | (D1041V) | | TOF |
| VII (1) | European | Compound | 14 | 2206G→A | Glu→Lys | 0/160 | MALDI- |
| | | heterozygote | | | (E696K) | | TOF |
| VIII (1) | European | Compound | 24 | 3221C→A | Ser→Phe | 0/180 | +StyI |
| | | heterozygote | | | (S1034F) | | |
| IX (1) | Arab | Compound | 19 | 2719G→A | Val→Met | 1/180 | +NlaIII |
| | | heterozygote | | | (V867M) | | |

^{a}Nucleotide position in CODANIN-1 cDNA accession number AF525398;
^{b}the mutation abolishes (−) or creates (+) a restriction site.

Eight additional codanin-1 mutations were identified in 7 French and in one English CDAI patients. Thus, 20 different mutations in 16 CDAI families were identified: 19/20 of the mutations are in exons 12-27 (6 in exon 14, 5 in exon 24, 2 in each of exons 12, 19 and one each in exons 6, 23 and 26). Such mutation clustering pattern suggests that these mutated exons are either more prone to mutation or that they encode essential functional domains. Furthermore, although nonsense mutations were found in two compound heterozygotes, no homozygotes for null-type mutations have been identified. This suggests that codanin-1 may have a unique and critical function, with very little redundancy.

BLOCKS defining 13 inter-species sequence conservation regions (FIG. 4, A-M). Subsequently, these BLOCKs were compared to all BLOCK database motifs, using the Local Alignment of Multiple Alignment (LAMA) method. A cluster of mutations around amino acids 1034-41 and 866-867 was noted. Similarities to two domains of the microtubule-associated protein MAP1B (FIG. 4, blocks C and E), and to two domains of fibrillar collagen triple helix repeats (FIG. 4, blocks A and G) were unveiled. Using CYRCA more remote, indirect similarities were further detected between the triple helix repeats blocks and synapsins blocks, a protein family known to be involved in binding diverse substrates including actin, spectrin, and microtubules. CYRCA is a method developed to identify Blocks of potentially similar function and structure appearing in different contexts. Based on the disease phenotype, which includes spongy heterochromatin and invagination of the nuclear membrane, and the protein similarity results, it is possible that codanin-1 might be intricated in nuclear membrane integrity by connecting the nuclear membrane and microtubules.

### Example 4

### Electronic-Database Information

GenBank (http://www.ncbi.nlm.nih.gov/Genbank/) was used to track CDAN1 locus, and Codanin-1 (and the corresponding accession numbers) as follows: KIAA0770 (AB018313), DKFZP564G2022 (NM_{—}015497), ZFP106 (NM022473), SNAP23 (NM_{—}003825), FLJ10460 (NM_{—}018097), FLJ23375 (NM_{—}024956), CCNDBP1 (NM_{—}012142), EPB42 (NM_{—}000119), TGM5 (NM_{—}004245), UBR1 (AF525401), TTBK (AF525400), FLJ008 (AF525397), CDANI (AF525398) and LOC146050 (AF525399).

Online Mendelian Inheritance in Man (OMIM; http://www.ncbi.nlm.nih.gov/Omim) was used to search for CDAI [MIM 224120], CDAII [MIM 224100], and CDAIII [MIM 105600].

BLAST (http://www.ncbi.nlm.nih.gov/BLAST/) was used to search for codanin-1 homologs and orthologs, as well as genetic markers.

Genomic browsers were utilized for defining known and unknown genes within the genomic interval: Ensemble (http://www.ensembl.org/, UCSC (http://genome.ucsc.edu/) and Celera (http:/www.celera.com/).

The following Protein motif servers were applied:

Blocks (http://bioinformatics.weizmann.ac.il/blocks/,

InterPro (http://www.ebi.ac.uklinterpro/scan.html),

PROSITE (http://npsa-pbil.ibcp.fr/cgi-bin/npsa automat.pl?page=npsa_prosite.html), and

Pfam (http://bioinformatics.weizmann.ac.il/Pfam/).

Local Alignment of Multiple Alignment (LAMA; http://bioinformatics.weizmann.ac.il/blocks-bin/LAMA_search.sh.) was launched for sequence motifs search. For gene prediction NIX (http://www.hgmp.mrc.ac.uk/NIX/) was applied. To detect signal peptides and transmembrane domains SignalIP (http://www.cbs.dtu.dk/services/SignalP/), PredictProtein (http:///cubic.bioc.columbia.edu/predictprotein/) and SOSUI (http://sosui.proteome.bio.tuat.ac.ip/sosuiframe0.html) were used.

### Example 5

### Cellular Location of Codanin-1

Use of polyclonal anti-codanin-1 antibodies indicates that codanin-1 protein may be located inside the cell nucleus. Without wishing to be bound by any particular concept or mechanism of action, these findings, and the cellular phenotype of the CDAI disease, suggest that codanin-1 may be involved in nuclear envelope integrity, conceivably related to microtubule attachments.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

### REFERENCES

Gasparini P, et al. (1997) Am. J. Hum. Genet 61:1112-1116
Higgins D G, et al. Methods Enzymol. 266:383-402
Iolascon A, et al. Blood 98:1258-1260
Lind L, et al., (1995) Hum. Mol. Genet 4:109-112
Pearson W R, Lipman D J. (1988) Proc Natl Acad Sci USA. 85:2444-8.
Pope S H, et al., (2000) Eur J Haematol 64:292-303
Sauer S, et al. (2002) Nucleic Acids Res. 30:e22
Shanske A, et al., (2001) Am J Med Genet 102:231-6
Tamary H, et al. (1996) Blood 87:1763-1770
Tamary H, et al. (1998) Am J Hum Genet 62:1062-1068

1. An erythrocyte differentiation factor comprising an amino acid sequence as set forth in at least one of SEQ ID NOS: 1-3, or a fragment, a mutant or a variant thereof.

2. The erythrocyte differentiation factor according to claim 1, obtained from a mammalian species.

3. The erythrocyte differentiation factor according to claim 1, obtained from a human source.

4. The erythrocyte differentiation factor according to claim 1, wherein said differentiation factor is glycosylated.

5. The erythrocyte differentiation factor according to claim 1, wherein said differentiation factor promotes the differentiation of hematopoietic stem cells to erythrocytes.

6. The erythrocyte differentiation factor according to claim 1, wherein said differentiation factor promotes proliferation of erythrocytes.

7. The erythrocyte differentiation factor according to claim 1, designated Codanin-1, deposited with the Genebank Database under Accession No. AF525398.

8. A pharmaceutical composition comprising an erythrocyte differentiation factor having an amino acid sequence as set forth in at least one of SEQ ID NOS: 1-3, or a fragment, a mutant or a variant thereof, and a pharmaceutically acceptable carrier.

9. An isolated nucleic acid encoding an erythrocyte differentiation factor, said isolated nucleic acid having a nucleotide sequence as set forth in SEQ ID NO: 4 (genomic clone AC090510), or a fragment, mutant or variant thereof.

10. The isolated nucleic acid according to claim 9, obtained from a mammalian species.

11. The isolated nucleic acid according to claim 9, obtained from a human source.

12. The isolated nucleic acid according to claim 9, wherein said differentiation factor promotes differentiation of hematopoietic stem cells to erythrocytes.

13. The isolated nucleic acid according to claim 9, wherein said differentiation factor promotes proliferation of erythrocytes.

14. The isolated nucleic acid according to claim 9, designated Codanin-1, deposited with the Genebank Database under Accession No. AF525398.

15. An expression vector comprising an isolated nucleic acid encoding an erythrocyte differentiation factor, said isolated nucleic acid having a nucleotide sequence as set forth in SEQ ID NO: 4 (genomic clone AC090510), or a fragment, mutant or variant thereof.

16. A method of promoting differentiation of hematopoietic stem cells to erythrocytes in vitro, comprising the step of culturing hematopoietic stem cells in a culture medium comprising an erythrocyte differentiation factor having an amino acid sequence as set forth in at least one of SEQ ID NOS: 1-3, or a fragment, mutant or variant thereof, in an amount effective to promote the differentiation of said hematopoietic stem cells to erythrocytes.

17. The method according to claim 16, wherein said differentiation factor is obtained from a mammalian species.

18. The method according to claim 16, wherein said differentiation factor is obtained from a human source.

19. The method according to claim 16, the culture medium further comprising an erythrocyte differentiation factor selected from the group consisting of: GMCSF, IL-3, and erythropoietin.

20. The method according to claim 16, wherein said differentiation factor is designated Codanin-1, deposited with the Genebank Database under Accession No. AF525398.

21. A method of producing erythrocytes in vitro, comprising the step of culturing hematopoietic stem cells in a culture medium comprising an erythrocyte differentiation factor having an amino acid sequence as set forth in at least one of SEQ ID NOS: 1-3, or a fragment, mutant or variant thereof, in an amount effective to induce differentiation of said hematopoietic stem cells, thereby producing said erythrocytes.

22. The method according to claim 21, wherein said differentiation factor is obtained from a mammalian species.

23. The method according to claim 21, wherein said differentiation factor is obtained from a human source.

24. The method according to claim 21, wherein said differentiation factor promotes the proliferation of said erythrocytes.

25. The method according to claim 21, wherein said differentiation factor is designated Codanin-1, the Genebank Database under Accession No. AF525398.

26. A method for producing erythrocytes in a subject, comprising the steps of:
a) removing hematopoietic stem cells from the subject;
b) contacting said hematopoietic stem cells with a culture medium comprising an erythrocyte differentiation factor derived from the same mammalian species as that of said subject, said differentiation factor having an amino acid sequence as set forth in at least one of SEQ ID NOS: 1-3, or a fragment, mutant or variant thereof;
c) culturing said cells in vitro, to produce a cellular preparation; wherein said culturing induces differentiation of said hematopoietic stem cells, thereby producing erythrocytes; and
d) administering said cellular preparation to the subject.

27. The method according to claim 26, wherein said differentiation factor is obtained from a human source.

28. The method according to claim 26, wherein said differentiation factor promotes the proliferation of erythrocytes.

29. The method according to claim 26, wherein said differentiation factor is designated Codanin-1, the Genebank Database under Accession No. AF525398.

30. A method of promoting differentiation of hematopoietic stem cells to erythrocytes in vitro, comprising the steps of:
a) transfecting a culture of hematopoietic stem cells with a vector comprising an isolated nucleic acid encoding an erythrocyte differentiation factor, said isolated nucleic acid having a nucleotide sequence as set forth in SEQ ID NO: 4 (genomic clone AC090510), or a fragment, mutant or variant thereof; and
b) expressing said differentiation factor in said hematopoietic stem cells; wherein said differentiation factor is expressed in an amount effective to promote the differentiation of said hematopoietic stem cells to erythrocytes.

31. The method according to claim 30, wherein said isolated nucleic acid is obtained from a mammalian species.

32. The method according to claim 30, wherein said isolated nucleic acid is obtained from a human source.

33. The method according to claim 30, wherein said differentiation factor promotes the proliferation of said erythrocytes.

34. The method according to claim 30, wherein said isolated nucleic acid is designated Codanin-1, the Genebank Database under Accession No. AF525398.

35. A method of producing erythrocytes in vitro, comprising the steps of:
a) transfecting a culture of hematopoietic stem cells with a vector comprising an isolated nucleic acid encoding an erythrocyte differentiation factor, said isolated nucleic acid having a nucleotide sequence as set forth in SEQ ID NO: 4 (genomic clone AC090510), or a fragment, mutant or variant thereof; and
b) expressing said differentiation factor in said hematopoietic stem cells; wherein said differentiation factor is expressed in an amount effective to induce differentiation of said hematopoietic stem cells, thereby producing said erythrocytes.

36. The method according to claim 35, wherein said isolated nucleic acid is obtained from a mammalian species.

37. The method according to claim 35, wherein said isolated nucleic acid is obtained from a human source.

38. The method according to claim 35, wherein said differentiation factor promotes the proliferation of said erythrocytes.

39. The method according to claim 35, wherein said isolated nucleic acid is designated Codanin-1, the Genebank Database under Accession No. AF525398.

40. A method for producing erythrocytes in a subject, comprising the steps of:
a) removing hematopoietic stem cells from the subject;
b) transfecting said hematopoietic stem cells with a vector comprising an isolated nucleic acid encoding an erythrocyte differentiation factor derived from the same mammalian species as that of said subject, said isolated nucleic acid having a nucleotide sequence as set forth in SEQ ID NO: 4, or a fragment, mutant or variant thereof;
c) expressing said differentiation factor in said hematopoietic stem cells;
d) culturing said cells in vitro, to produce a cellular preparation; wherein said culturing induces differentiation of said hematopoietic stem cells, thereby producing erythrocytes; and
e) administering said cellular preparation to the subject.

41. The method according to claim 40, wherein said isolated nucleic acid is obtained from a human source.

42. The method according to claim 40, wherein said differentiation factor promotes the proliferation of said erythrocytes.

43. The method according to claim 40, wherein said isolated nucleic acid is designated Codanin-1, the Genebank Database under Accession No. AF525398.

44. A method for treating anemia in a subject, comprising administering to the subject a therapeutically effective amount of an erythrocyte differentiation factor derived from the same mammalian species as that of said subject, said differentiation factor having an amino acid sequence as set forth in at least one of SEQ ID NOS: 1-3, or a fragment, mutant or varian thereof.

45. The method according to claim 44, wherein said differentiation factor is obtained from a human source.

46. The method according to claim 44, wherein said differentiation factor is designated Codanin-1, the Genebank Database under Accession No. AF525398.

47. A method for treating anemia in a subject, comprising administering to the subject a pharmaceutical composition comprising a therapeutically effective amount of an erythrocyte differentiation factor derived from the same mammalian species as that of said subject, said differentiation factor having an amino acid sequence as set forth in at least one of SEQ ID NOS: 1-3, or a fragment, mutant or variant thereof.

48. The method according to claim 47, wherein said differentiation factor is obtained from a human source.

49. The method according to claim 47, wherein said differentiation factor is designated Codanin-1, the Genebank Database under Accession No. AF525398.

50. The methods according to claim 49, wherein the pharmaceutical composition further comprises an erythrocyte differentiation factor selected from the group consisting of:
GMCSF, IL-3, and erythropoietin.

51. A method for treating anemia in a subject, comprising administering to the subject a pharmaceutical composition comprising a construct encoding an erythrocyte differentiation factor derived from the same mammalian species as that of said subject, said differentiation factor having a polynucleotide sequence as set forth in SEQ ID NO: 4, or a fragment, mutant or variant thereof.

52. The method according to claim 51, wherein said differentiation factor is obtained from a human source.

53. The method according to claim 51, wherein said differentiation factor is designated Codanin-1, the Genebank Database under Accession No. AF525398.

54. A method for diagnosing anemia in a subject, comprising the step of analyzing in the subject the gene encoding an erythrocyte differentiation factor derived from the same mammalian species as that of said subject, said differentiation factor having a polynucleotide sequence as set forth in SEQ ID NO: 4, or a fragment, mutant or variant thereof.

55. The method according to claim 54, wherein said differentiation factor is obtained from a human source.

56. The method according to claim 54, wherein said differentiation factor is designated Codanin-1, the Genebank Database under Accession No. AF525398.

57. A host cell comprising a vector comprising a polynucleotide sequence as set forth in SEQ ID NO: 4, or a fragment, mutant or variant thereof.

58. The host cell of claim 57, wherein the vector further comprises a promoter operatively linked to the polynucleotide sequence as set forth in SEQ ID NO: 4, or a fragment, mutant or variant thereof.

59. The host cell according to claim 57, wherein the vector further comprises at least one selectable marker.

60. The host cell according to claim 57, wherein the vector is a plasmid or a virus.

61. The host cell according to claim 60, wherein the virus is selected from the group consisting of: adenoviruses, retroviruses or lentiviruses.

62. The host cell according to claim 57, wherein the cell is eukaryotic.

63. The host cell according to claim 62, wherein the cell may be selected from the group consisting of: human embryonic stem cell and human mesenchymal cell.

64. An antibody capable of binding to an erythrocyte differentiation factor comprising an amino acid sequence as set forth in at least one of SEQ ID NOS: 1-3, or a fragment, a mutant or a variant thereof.

65. The antibody according to claim 64, selected from the group consisting of: a full length antibody, an antibody having a human immunoglobulin constant region, a monoclonal IgG, a monoclonal IgG of subclasses γ1 or γ4, a single chain antibody, an antibody fragment, a labeled antibody, an immobilized antibody, an antibody conjugated with a heterologous compound.

66. The antibody according to claim 64, capable of binding to an erythrocyte differentiation factor designated Codanin-1, deposited with the Genebank Database under Accession No. AF525398.

67. A method for diagnosing Dyserythropoietic anemias type I in a subject, comprising contacting the antibody according to claim 64 with a fluid sample retrieved from the subject.


SEQUENCE LISTING (4 sequences)

SEQ ID NO: 1 - protein, 1226 aa, Homo sapiens
MAAVLESLLREEVSVAAVVRWIARSTQGSEVTAGEAAAVSSFGAWRKEFVRSLLTFLREQSSRVLPQGPPTPPKTRGASAACPGRPGGPPRGTRGARSQLFPPTEAQSTAAEAPLARRGGRRRGPGPARERGGRGLEEGVSGESLPGAGGRRLRGSGSPSRPSLTLSDPPNLSNLEEFPPVGSVPPGPTGETQLSCRRWVPRRLWGVSHSSSALRSGAPGCRTKPSRRINPTPVSEERSLSKPKTCFTSPPISCVPSSKQLQQSPTPTCPTPELGSPLPSRTGSLTDEPADPARVSSRQRLELVALVYSSCIAENLVPNLFLELFFVFQLLTARRMVTAKDSDPELSPAVLDSLESPLFQSIHDCVFFAVQVLECHFQVLSNLDKGTLKLLAENERLLCFSPALQGRLRAAYEGSVAKVSLVMPPSTQAVSFQPETDNRANFSSDRAFHTFKKQRDVFYEVLREWEDHHEEPGWDFEKGLGSRIRAMMGQLSAACSHSHFVRLFQKQLLQMCQSPGGAGGTVLGEAPDVLSMLGADKLGRLWRLQERLMAPQSSGGPCPPPTFPGCQGFFRDFILSASSFQFNQHLMDSLSLKIQELNGLALPQHEPNDEDGESDVDWQVERKQFAVVLLSLRLLAKFLGFVAFLPYRGPEPPPTGELQDSILALRSQVPPVLDVRTLLQRGLQARRAVLTVPWLVEFLSFADHVVPLLEYYRDIFTLLLRLHRCLVLSQESEGKMCFLNKLLLLAVLGWLFQIPTVPEDLFFLEEGPSYAFEVDTVAPEHGLDNAPVVDQQLLYTCCPYIGELRKLLASWVSGSSGRSGGFMRKITPTTTTSLGAQPSQTSQGLQSQLAQAFFHNQPPSLRRTVEFVAERIGSNCVKHIKATLVADLVRQAESLLQEQLVTQGEEGGDPAQLLEILCSQLCPHGAQALALGREFCQRKSPGAVRALLPEETPAAVLSSAENIAVGLATEKACAWLSANITALIRREVKAAVSRTLRAQGPEPAARGERRGCSRACEHHAPLPSHLISEIKDVLSLAVGPRDPDEGVSPEHLEQLLGQLGQTLRCRQVLCPPAEQHLAKCSVELASLLVADQIPILGPPAQYRLERGQARRLLHMLLSLWKEDFQGPVPLQLLLSPRNVGLLADTRPREWDLLLFLLRELVEKGLMGRMEIEACLGSLHQAQWPGDFAEELATLSNLFLAEPHLPEPQLRACELVQPNRGTVLAQS

SEQ ID NO: 2 - protein, 1239 aa, Mus musculus
MAAVLESLLREEVPVAAAVRWIARSTPSSEGGRSERTSASAEGIRAVPAELPEGAEQPGPPAGPLNPRQDPSRLGSLGAPARGGRGARSQLFPAAEPLSAAAEAPLARRAGRRRGPGPGPSRERGGRGGESPPWAGGRKPKGSGSPGSPRLSLSDPPNLSNLEEFPPVGTVPPGSAGRTKPSRRINPTPVSEERSLSKPKTCFTSPPISCVPSTKQLQQSPTPASPIPESGSPVPSRTGNLTAEPADPARVSSRQRLELVALIYSSCIAENLVPNLFLELFFVLQLLTARRMVATKDSDLESSQGALDSLDTPLFRSIHDCVFFAVQVLEHQFQVLSYLDKGTLKLLAENERLLCFSPALQGRLRAAYEGSVAKVSLVIPPSAQAVSFQPETDNRANFSSDRAFHTFKKQRDVFYEVLREWEDHHEEPSWDFEKGLGSRIRAMMGQLSAACSHSHFVRLFQKQLLQMCQSPGGAGGSVLGEAPDVLNMLGADKLGRLRQLQERLIAPQSSGGPCPPPTFPGCQGFFRDFIMSASFHFNQHLMDSLSLKIRELNGLRLPQHEPGDEDGESDMDWQVERRQFAVVLLSLRLLAKFLGFVAFLPYRGHEPPPTRELQDSILALRSQVPPVLDIRALLQQGLWARRAVLTVPWLVEFLSFADHIVPLLDYYRSVFTLLLRLHRELVLSKENEGEMCFLNKLLLLAVLGWLFQVPTVPEDLFFLEDGQVDAFEVTTTASEHGLDSVPVVDQQLLYTCCPYIGELRKLLASWVSGSSGRSGGFVRKITPTTTSSLGALPLQTSQGLQVRAKDQETGGQLGREAEGSPHGTTTPASTSCQAQLAEAFFHNQPPSLRRTVEFVAERIGSNCVKHIKTLVADLVHQAESLLQEQLVARGQEGGDPAQLLESLCSQLCPHGAQALTQGREFCQRKSPTAVRALLPEETPAAVLSSAENIAVGLATEKACSWLSANITLIRREVKAAVSRMLRAQGPEPTARVERRGCSRACEHHAPLPSHLISEIKSFCLSPLAGSHPKPSFWHSGMKFHHCSLQDVLSLAAGPRDPEEGVSPEHLEQLLNQMGQSLRCRQVLCPTAEQHLAKCSVELASLLADQIPILGPPTQHRLERGHARRLLHMLLSLWKDDFQGPVPLQLLLSPRNVGLLADTRPREWDLLLFLLRELVEKDLMGHLEIEACLGRLNEAQWPGDFSEELSTLFRLFLAEPHLLEPQLRACELMQPNRGTVLAQS

SEQ ID NO: 3 - protein, 765 aa, Fugu rubripes
SAGSERRSGQRFSLGDYVVSPPDLQPSPSFQLQRGRRRGAGNLPTGGPGRGGRGGHHGDENGGRRSSRGGGGYSRISEQVSPPSVGQLNLSNLEDFPPVGSSPISPPSRRINPTPVSGERPHSKTKTCFTSTPFENLVPNIFLELFFVMQLLTSRSTHTHDDNKQERLISHLDKCTLRLLAENERVASFSPELRDYLSQAQDKSTAKQSHSSLLLTIAPTSTVTGLSTPSKNNRDIFYEVLREWEDFHKEPSWSFDAALSSKIMCKGPPVNSSPGDAPDADLLGMLGADSLGRLKRLEERLIQPYGVIGPCPPPSFPGHQEFFRDFIQAASQLNQHLQDSLCQQLLQLDEVSIQSPPASSGKEEEEEEDGDMEQQRFNIFDVTCRRMLLGRGGEMCYLNKLLMVSVLGWLFQVINSFSTPVVLFSVSGAWIQPLPSSVLGDEQNTNVQSNRLFHTNAGEFRKLLAAFVSGSTAKSGGIIRKITPTSAELRDASTPNRSQQKLEVVDLEQAFFHNQPPSLRRTVEFVAERVGSNAVKHMKRFCSEKSPRAIRILLPDETSSAVALIRREWKSRYDRVMKEVGGPPALDLCDVVREERVTTPKRKQGSQRAASCPPDCPHTAPLPSDVFIELKVCSFVAVQEMLSVAVGPRTEEELPAACQVEALLQRLRDTLVCRKFSTVVSEQMLLSSTVLLACKLVSAELPLASALERGDGVVAGPGSGSEVPVRALLVQLVELWEKDCCSSAPLHLLFTPLTVTAVLNASDTE

SEQ ID NO: 4 - DNA, 4725 bp, Homo sapiens
ccgggtccctccccatcccgcgacccaacggtcgttccccgccgccggccccgcccctccctggccgcccgcggcgcgccggcgcctgcgcagtcgccccagagcgccccgaccgggatggcggccgttttggagtcgctgctgcgagaagaggtgtcggtcgcagccgtcgtgcggtggatcgcgcgcagcacccagggttcggaggtaacagctggggaggcggccgcggtgagctcattcggggcctggcggaaagaattcgtacgttccctgttgaccttcctgagggagcagagcagccgggtcctcccgcaggggcccccgaccccgcccaagacccggggcgcctcggcagcttgcccagggaggccggggggcccgccgcggggaacccgcggggcgcgcagccagcttttccctccgaccgaggcccagagcaccgctgccgaggcccctctggcccgccgcgggggcaggaggcggggcccggggccggcccgcgagcgtggaggccgcggcctggaggagggggtcagcggggagagcctgcccggagccgggggccggaggcttaggggctctggcagccccagccgccccagcctcacgctgtctgatccgccaaacctcagcaacctggaggagttccctcccgtaggctcggttccccccggccctacaggtgagactcagctctcatgcaggagatgggtaccacgaaggctctggggagtcagtcattcgagctcggcgctccgcagtggagcgccaggatgcaggacgaagccttctcgcaggatcaacccaactccggtgagcgaagagcggtcactctccaagcccaagacctgcttcacctcacccccaatcagctgtgtccccagctctaagcagctgcagcagtcacctacccccacctgtcccaccccagaattggggtcgcccctccccagccggacaggaagcctcacagatgaacctgcagaccctgccagagtgtcttcccgccagcgcctggagcttgtagcccttgtttactcctcgtgcattgctgagaacctggtaccaaacctcttcttggagcttttcttcgtctttcagctcctcactgcccggaggatggtgactgccaaggacagcgaccctgaactaagtccagctgtcctagattccctggaaagtccactgttccaaagcatccacgattgtgtcttctttgcagtgcaggttttggagtgtcactttcagtttctttccaacctggacaaagggaccttgaagctgctggctgagaatgagcggctgctgtgcttctcaccagctctgcaaggccgccttcgagctgcctatgagggcagtgttgccaaggtctctctggtgatgcctccctctactcaagctgtctcctttcagccagagactgacaatcgtgccaacttctccagtgaccgagcctttcatacttttaagaaacagagggatgtgttttatgaggtgctgcgagagtgggaagatcaccatgaggagcctggctgggattttgagaagggcttgggcagcagaatcagggccatgatgggtcagctctccgcagcctgcagccacagccactttgttcggcttttccaaaaacaactactccagatgtgtcagagccctggtggtgctgggggcaccgtcttgggcgaggccccagatgtgttgagtatgctgggagctgacaagctggggcggttgtggcgcctacaggaacggcttatggctcctcagagcagtggggggccctgcccaccccccaccttcccaggctgtcaaggcttctttagggacttcatccttagtgccagcagcttccagtttaaccagcatctcatggacagtctgagcttgaagatccaggagctcaatggtcttgccctgccccagcatgagcccaatgatgaagacggggagtcagacgtagactggcagggtgagcggaagcaatttgctgtggtgcttcttagcctgagacttttggctaaattcctgggctttgtggctttcctgccataccgggggcctgaacctcccccgaccggtgagcttcaggactccattctggccctcaggagccaggtccctccggtcctggatgtgcggactctgctgcagcgagggctgcaggcccgccgggcggtgctcaccgtgccctggctggtggagtttctctcctttgctgaccatgttgttcccttgctggaatattaccgggacatcttcactctcctgctgcgcctgcaccggtgcttggtgttgtcgcaggagagtgaggggaagatgtgtttcctgaacaagctgctgctacttgctgtcctgggctggcttttccagattcccacagtccctgaggacttgttctttctggaagagggtccctcatatgcctttgaggtggacacagtagccccagagcatggcttggacaatgcgcctgtggtggaccagcagctgctctacacctgctgcccctacatcggagagctccggaaactgctcgcttcgtgggtgtcaggcagtagtggacggagtgggggcttcatgaggaaaatcacccccaccactaccaccagcctgggagcccagccttcccagaccagccaggggctgcaggcacagctcgcccaggcctttttccacaaccagccgccctccttgcgccggaccgtagagttcgtggcagaaagaattggatcaaactgtgtcaaacatatcaaggctacactggtggcagatctggtgcgccaggcagagtcacttctccaagagcagctggtgacacagggagaggaagggggagacccagcccagctgttggagatcttgtgttcccagctgtgccctcacggggcccaggcattggccctggggcgggagttctgtcaaaggaagagccctggggctgtgcgggcgctgcttccagaggagaccccggcagccgttctgagcagtgcagagaacattgctgtggggcttgcaacagagaaagcctgtgcttggctgtcagccaacatcacagcactgatcaggagggaggtgaaagcagcagtgagtcgcacacttcgagcccagggtcctgaacctgctgcccggggggagcggaggggctgctcccgcgcctgtgagcaccatgctcccctcccctcccacctcatctccgagataaaagacgtgctctccttggccgtggggccacgggaccctgacgagggagtctccccagagcatctggaacagctcctaggccagctgggccagacgctgcggtgccgccagttcctgtgcccacctgctgagcagcatctggcaaagtgctctgtggagttagcttccctcctcgttgcagatcaaattcctatcctagggcccccggcacagtacaggctggagagagggcaggctcgaaggcttctgcacatgctgctttccttgtggaaggaagactttcaggggccggttccgctgcagctgctgctgagcccaagaaatgtggggcttctggcagacacaaggccaagggagtgggacttgctgctattcttgctacgggagctggtggagaagggtctgatgggacggatggagatagaggcctgcctgggcagcctccaccaggcccagtggccaggggactttgctgaagaattagcaacactgtctaatctgtttctagccgagccccacctgccagaaccccagctaagagcctgtgagttggtgcagccaaaccggggcactgtgctggcccagagctagggctgagaagtggccctgccttgggcattgcaccagaaccctggacccccgcctcacgaggaggcccaagtgcccaatgcagaccctcactggttggggtgtagctgggtctacagtcagacttcctgctctaagggtgtcactgcctggcatcccaccacgcgaatcctagaggaaggagagttggcctgatttgggattatggcagaaaagtccagagatgccagtcctggagtagaagaggtggtgtttgtttatctcttggatactaaatgaaatgaggtgtgtgggcttgtcaacacagaattcaagcctcatttgctatcccagcatctcttaaaactttgtagtcttggaattcatgacagaggcaaatgactcctgcttaacttatgaagaaagttaaaacatgaatcttgggagtctacattttcttatcaccaggagctggactgccatctccttataaatgcctaacacaggccgggtctggtggctcatgcctgtaatcccagcactttgagaggcctgaggtcggcggactgcctgaggtcaggaattcaagaccagcctggccaacatggcaaaaccccatctctactaaaaataaaaaaattattagctgggcatggtggtgtgtgcctgtaatcccagctactcaggaggatgaggcaggagacctgcttgaacctggaggtggaggttgcagtgagccgaggtcgcaccactgcactccagtctgggtaacagagcgagactttctagaaaaagcctaacaaacagataaggtaggactcaaccaactgaaacctgactttccccctgtaccttcagcccctgtgcaggtagtaacctcttgagacctctccctgaccagggaccaagcacagggcatttagagctttttagaataaactggttttcttt